(19) United States
(12) Patent Application Publication
(10) Pub. No.: US 2021/0154430 A1
(43) Pub. Date: May 27, 2021

(54) SYSTEMS AND METHODS FOR PREDICTING AND PREVENTING MOTION SICKNESS

(71) Applicant: MOBILEX LTD, Savyon (IL)

(72) Inventors: Ron LEV, Modiin - Reut (IL); Eyal FRIED, Haifa (IL); Jonathan GIRON, Tzur Yitzhak (IL)

(21) Appl. No.: 17/259,168

(22) PCT Filed: Jul. 4, 2019
(86) PCT No.: PCT/IL2019/050743

§ 371 Date: Jan. 10, 2021

Related U.S. Application Data
(60) Provisional application No. 62/695,861, filed on Jul. 10, 2018.
(60) Provisional application No. 62/751,634, filed on Oct. 28, 2018.

(51) Int. Cl. A61M 21/00; G16H 50/30; G16H 50/70; G16H 40/67; G16H 10/60; G16H 50/20; G06Q 50/30; G07C 5/08

(52) U.S. Cl. CPC A61M 21/00; A61M 2021/0022; G16H 50/70; G16H 40/67; G16H 10/60; G16H 50/20; G06Q 50/30; G07C 5/08; A61M 2021/0011; A61M 2021/005; A61M 2021/0038; A61M 2021/0066; A61M 2205/3313; A61M 2205/3375; A61M 2205/3368; A61M 2205/3584; A61M 2205/507; A61M 2230/06; A61M 2230/10; A61M 2230/42; A61M 2230/50; A61M 2230/60; A61M 2205/3303; G16H 50/30

(57) ABSTRACT

A system, the system comprising: a controller; and a memory configured to store data used by the controller, the controller being configured to: provide at least one model indicative of a relationship between first values of one or more physiological parameters of a group of one or more persons and a first probability of the persons within the group to get motion sickness within one or more given time periods; obtain, from one or more sensors, second values of the physiological parameters, the second values being readings obtained by the sensors from one or more given persons; estimate, based on the second values and on the model, a second probability of each of the given persons to get motion sickness; and provide one or more instructions to perform one or more motion sickness prevention measures for at least one of the given persons upon the second probability of the at least one of the given persons exceeding a threshold, to at least reduce the second probability.

### TECHNICAL FIELD

The invention relates to systems and methods for predicting and preventing motion sickness.

### BACKGROUND

Humans are at a heightened risk of getting Motion Sickness when using the new technologies of driverless (autonomous) vehicles and Virtual Reality (VR), separately and/or in combination, since use of these technologies by humans often results in a dissonance between motion that they feel and motion that they see, and since humans are at a heightened risk of getting motion sickness when there is a dissonance between the motion that they feel and the motion that they see.

Specifically, the vestibular system (i.e., ears) of a passenger in a driverless (autonomous) vehicle (or in human-driven/operated vehicle) may: (a) feel vehicle movement without the visual system (i.e., eyes) of the passenger predicting the vehicle movement that is felt, and (b) feel an orientation of the vehicle without the visual system of the passenger sensing, by sight, the orientation of the vehicle. This may occur, for example, due to the sides of the vehicle being opaque or due to internal content consumption by the passenger.

In addition, the visual system of the passenger may predict movement of the vehicle and sense orientation of the vehicle without the vestibular system of the passenger feeling the movement and orientation of the vehicle, for example, due to typical “Living Space” sittings at different angles, especially in dynamic acceleration situations.

Moreover, the visual system of a person, whether or not a vehicle passenger, may see movement when viewing visual content (e.g. through an in-vehicle entertainment/infotainment system), and/or virtual reality (VR) content, while the vestibular system of the person does not feel the movement that is seen.

With respect to the VR industry, VR applications and the VR industry are currently handicapped by severe motion sickness symptoms for a large section of the users (25-40%) due to a dissonance between motion felt and motion seen.

In view of the foregoing, the number of cases of motion sickness can be expected to increase in accordance with an increase in the use of the technologies of driverless (autonomous) vehicles and VR, alone or in combination. Accordingly, it is critical that there be operational systems that are capable of preventing the onset of motion sickness in users of driverless vehicles and VR in order to enable the enhanced use of these technologies.

Currently, there are no successful operational systems to prevent the onset of motion sickness in persons, much less predict motion sickness and perform prevention measures accordingly.

There is thus a need in the art for new systems and methods for prediction and prevention of motion sickness.

### General Description

In accordance with a first aspect of the presently disclosed subject matter, there is provided a system, the system comprising: a controller; and a memory configured to store data used by the controller, the controller being configured to: (a) provide at least one model indicative of a relationship between first values of one or more physiological parameters of a group of one or more persons and a first probability of the persons within the group to get motion sickness within one or more given time periods; (b) obtain, from one or more sensors, second values of the physiological parameters, the second values being readings obtained by the sensors from one or more given persons; (c) estimate, based on the second values and on the model, a second probability of each of the given persons to get motion sickness; and (d) provide one or more instructions to perform one or more motion sickness prevention measures for at least one of the given persons upon the second probability of the at least one of the given persons exceeding a threshold to at least reduce the second probability.

In some cases, the controller is further configured to repeat steps (b) to (d) a plurality of times over a time period.

In some cases, the controller is further configured to perform the motion sickness prevention measures, thereby at least reducing the second probability.

In some cases, the motion sickness prevention measures are selected based on results of motion sickness prevention measures previously performed for the at least one of the given persons associated with the second probability exceeding the threshold.

In some cases, the motion sickness prevention measures include one or more of: providing a first subliminal or explicit visual cue that is indicative of at least a global horizon to the at least one of the given persons associated with the second probability exceeding the threshold; displaying the content provided to the at least one of the given persons associated with the second probability exceeding the threshold in infinity; projecting encoring lights to be viewed by the at least one of the given persons associated with the second probability exceeding the threshold; projecting flashing lights towards the at least one of the given persons associated with the second probability exceeding the threshold; reducing a sensitivity of a vestibular system of the at least one of the given persons associated with the second probability exceeding the threshold; influencing the vestibular system of the at least one of the given persons associated with the second probability exceeding the threshold by transmitting ultrasound waves; inducing vibrations on at least part of the at least one of the given persons associated with the second probability exceeding the threshold; providing haptic feedback to the at least one of the given person associated with the second probability exceeding the threshold; playing stress reducing audio or visual recordings for the at least one of the given persons associated with the second probability exceeding the threshold; generating an automatic conversation with the at least one of the given persons associated with the second probability exceeding the threshold; inducing or enhancing pressure on at least part of the at least one of the given persons associated with the second probability exceeding the threshold; moving a chair or seat of the at least one of the given persons associated with the second probability exceeding the threshold; generating one or more blasts of wind cues from different positions surrounding the at least one of the given persons associated with the second probability exceeding the threshold towards the at least one of the given persons; or generating one or more sound cues from different positions surrounding the at least one of the given persons associated with the second probability exceeding the threshold towards the at least one of the given persons.

In some cases, the first subliminal visual cue is provided at a varying display rate, based on the second probability.

In some cases, the given persons are vehicle passengers in a vehicle driving along a driving route.

In some cases, the vehicle is a driverless autonomous vehicle.

In some cases, the estimate is also based on at least one of: the driving route of the vehicle, driving conditions along the driving route of the vehicle, a current speed of the vehicle, a predicted speed of the vehicle, a temperature within the vehicle, a type of vehicle, or information of accelerations of the vehicle in at least one axis.

In some cases, the motion sickness prevention measures include one or more of: stopping the vehicle; slowing down the vehicle; limiting the vehicle's accelerations in at least one axis of movement; limiting the vehicle's speed; calculating a new driving route for the vehicle; providing a second subliminal or explicit indication of a predicted next turn of the vehicle along the driving route to the at least one of the given persons associated with the second probability exceeding the threshold; correlating visual content provided to the at least one of the given persons associated with the second probability exceeding the threshold to the driving route, giving rise to correlated content; changing the vehicle's windows transparency; changing the vehicle's internal lighting strength; changing a temperature within the vehicle; eliminating one or more induction vibrations within a frequency range of 240 to 260 Hz that surround the at least one of the given persons associated with the second probability exceeding the threshold; generating one or more blasts of wind cues correlated with the correlated content towards the at least one of the given persons associated with the second probability exceeding the threshold; or generating one or more sound cues correlated with the correlated content towards the at least one of the given persons associated with the second probability exceeding the threshold.

In some cases, the given persons are consumers of Virtual Reality (VR) content wearing a VR headset.

In some cases, the motion sickness prevention measures include one or more of: stopping projection of content in the VR headset worn by the at least one of the given persons associated with the second probability exceeding the threshold; changing a temperature within a room in which the at least one of the given persons associated with the second probability exceeding the threshold is located; providing haptic feedback to the at least one of the given persons associated with the second probability exceeding the threshold, using straps of the VR headset; changing a transparency of the VR headset; changing a light intensity of the VR headset; moving a chair of the at least one of the given persons associated with the second probability exceeding the threshold to at least partially mimic movements made within the content displayed in the VR headset; generating one or more blasts of wind cues correlated with the content displayed in the VR headset towards the at least one of the given persons associated with the second probability exceeding the threshold, or generating one or more sound cues correlated with the content displayed in the VR headset towards the at least one of the given persons associated with the second probability exceeding the threshold.

In some cases, the at least one model is person-specific or one or more of: gender specific, race-specific, or age-range specific.

In some cases, the physiological parameters include at least one, or a combination of two or more of: heart rate, heart rate variability, sweat level, respiration rate, skin color, pupil dilation, blood pressure, muscle tension, body temperature, eye gaze movement, microexpression, body movement, electroencephalography (EEG) readings, voice phonation, voice pitch, voice loudness, or voice rate.

In some cases, the sensors include at least one of: one or more contact sensors, or one or more proximity or remote sensors such as, but not limited to: lasers, radars, cameras, infrared (IR) sensors, Terahertz radiation sensors, ultrasound sensors, or acoustic sensors.

In accordance with a second aspect of the presently disclosed subject matter, there is provided a system, the system comprising: a controller; and a memory configured to store data used by the controller, the controller being configured to: obtain (a) information of a planned driving route of a driverless autonomous vehicle and (b) an indication of a location of the driverless autonomous vehicle on the planned driving route; and provide one or more instructions to perform one or more motion sickness prevention measures, based on the information and on the indication.

In some cases, the controller is further configured to perform the motion sickness prevention measures.

In some cases, the provide is also based on past information of motion sickness experienced by one or more passengers riding along the planned driving route.

In some cases, at least one of the passengers is a current passenger riding in the driverless autonomous vehicle on the planned driving route.

In some cases, the provide is also based on vehicle movement information obtained from one or more sensors monitoring movements of the driverless autonomous vehicle.

In some cases, the motion sickness prevention measures include one or more of: providing a first subliminal or explicit visual cue that is indicative of a global horizon to at least one passenger of the driverless autonomous vehicle; providing a second subliminal or explicit visual cue that is indicative of a predicted next turn of the vehicle along the driving route to the at least one passenger; correlating visual content provided to the at least one passenger to the driving route, giving rise to correlated content; changing the vehicle's windows transparency; displaying the content provided to the at least one passenger in infinity; changing the vehicle's internal lighting strength; changing a temperature within the vehicle; projecting encoring lights to be viewed by the at least one passenger; projecting flashing lights towards the at least one passenger; reducing a sensitivity of the vestibular system of the at least one passenger; influencing the vestibular system of the at least one passenger by transmitting ultrasound waves; eliminating one or more induction vibrations within a frequency range between 240 Hz and 260 Hz that surround at the at least one passenger; inducing vibrations on at least part of the at least one passenger; providing haptic feedback to the at least one passenger; playing one or more stress reducing audio or visual recordings to the at least one passenger; generating an automatic conversation with the at least one passenger; inducing or enhancing pressure on at least part of the at least one passenger; moving a chair of the at least one passenger; generating one or more blasts of wind cues from different positions surrounding the at least one passenger towards the at least one passenger; generating one or more sound cues from different positions surrounding the at least one passenger towards the at least one passenger; stopping the driverless autonomous vehicle; slowing down the driverless autonomous vehicle; limiting the driverless autonomous vehicle's accelerations in at least one axis of movement; calculating a new driving route for the driverless autonomous vehicle; generating one or more blasts of wind cues correlated with the correlated content from different positions surrounding the at least one passenger towards the at least one passenger; or generating one or more sound cues correlated with the correlated content from different positions surrounding the at least one passenger towards the at least one passenger.

In accordance with a third aspect of the presently disclosed subject matter, there is provided a system, the system comprising: a controller; and a memory configured to store data used by the controller, the controller being configured to: obtain an orientation measurement from at least one orientation measurement sensor of a driverless autonomous vehicle; and display content including a subliminal or explicit visual cue that is indicative of a global horizon aligned with the orientation measurement to at least one passenger of the driverless autonomous vehicle.

In accordance with a fourth aspect of the presently disclosed subject matter, there is provided a method, comprising: (a) providing at least one model indicative of a relationship between first values of one or more physiological parameters of a group of one or more persons and a first probability of the persons within the group to get motion sickness within one or more given time periods; (b) obtaining, from one or more sensors, second values of the physiological parameters, the second values being readings obtained by the sensors from one or more given persons; (c) estimating, based on the second values and on the model, a second probability of each of the given persons to get motion sickness; and (d) providing one or more instructions to perform one or more motion sickness prevention measures for at least one of the given persons upon the second probability of the at least one of the given persons exceeding a threshold to at least reduce the second probability.

In some cases, the method further comprises repeating steps (b) to (d) a plurality of times over a time period.

In some cases, the method further comprises performing the motion sickness prevention measures, thereby at least reducing the second probability.

In some cases, the motion sickness prevention measures are selected based on results of motion sickness prevention measures previously performed for the at least one of the given persons associated with the second probability exceeding the threshold.

In some cases, the motion sickness prevention measures include one or more of: providing a first subliminal or explicit visual cue that is indicative of at least a global horizon to the at least one of the given persons associated with the second probability exceeding the threshold; displaying the content provided to the at least one of the given persons associated with the second probability exceeding the threshold in infinity; projecting encoring lights to be viewed by the at least one of the given persons associated with the second probability exceeding the threshold; projecting flashing lights towards the at least one of the given persons associated with the second probability exceeding the threshold; reducing a sensitivity of a vestibular system of the at least one of the given persons associated with the second probability exceeding the threshold; influencing the vestibular system of the at least one of the given persons associated with the second probability exceeding the threshold by transmitting ultrasound waves; inducing vibrations on at least part of the at least one of the given persons associated with the second probability exceeding the threshold; providing haptic feedback to the at least one of the given person associated with the second probability exceeding the threshold; playing stress reducing audio or visual recordings for the at least one of the given persons associated with the second probability exceeding the threshold; generating an automatic conversation with the at least one of the given persons associated with the second probability exceeding the threshold; inducing or enhancing pressure on at least part of the at least one of the given persons associated with the second probability exceeding the threshold; moving a chair or seat of the at least one of the given persons associated with the second probability exceeding the threshold; generating one or more blasts of wind cues from different positions surrounding the at least one of the given persons associated with the second probability exceeding the threshold towards the at least one of the given persons; or generating one or more sound cues from different positions surrounding the at least one of the given persons associated with the second probability exceeding the threshold towards the at least one of the given persons.

In some cases, the first subliminal visual cue is provided at a varying display rate, based on the second probability.

In some cases, the given persons are vehicle passengers in a vehicle driving along a driving route.

In some cases, the vehicle is a driverless autonomous vehicle.

In some cases, the estimating is also based on at least one of: the driving route of the vehicle, driving conditions along the driving route of the vehicle, a current speed of the vehicle, a predicted speed of the vehicle, a temperature within the vehicle, a type of vehicle, or information of accelerations of the vehicle in at least one axis.

In some cases, the motion sickness prevention measures include one or more of: stopping the vehicle; slowing down the vehicle; limiting the vehicle's accelerations in at least one axis of movement; limiting the vehicle's speed; calculating a new driving route for the vehicle; providing a second subliminal or explicit indication of a predicted next turn of the vehicle along the driving route to the at least one of the given persons associated with the second probability exceeding the threshold; correlating visual content provided to the at least one of the given persons associated with the second probability exceeding the threshold to the driving route, giving rise to correlated content; changing the vehicle's windows transparency; changing the vehicle's internal lighting strength; changing a temperature within the vehicle; eliminating one or more induction vibrations within a frequency range of 240 to 260 Hz that surround the at least one of the given persons associated with the second probability exceeding the threshold; generating one or more blasts of wind cues correlated with the correlated content towards the at least one of the given persons associated with the second probability exceeding the threshold; or generating one or more sound cues correlated with the correlated content towards the at least one of the given persons associated with the second probability exceeding the threshold.

In some cases, the given persons are consumers of Virtual Reality (VR) content wearing a VR headset.

In some cases, the motion sickness prevention measures include one or more of: stopping projection of content in the VR headset worn by the at least one of the given persons associated with the second probability exceeding the threshold; changing a temperature within a room in which the at least one of the given persons associated with the second probability exceeding the threshold is located; providing haptic feedback to the at least one of the given persons associated with the second probability exceeding the threshold, using straps of the VR headset; changing a transparency of the VR headset; changing a light intensity of the VR headset; moving a chair of the at least one of the given persons associated with the second probability exceeding the threshold to at least partially mimic movements made within the content displayed in the VR headset; generating one or more blasts of wind cues correlated with the content displayed in the VR headset towards the at least one of the given persons associated with the second probability exceeding the threshold, or generating one or more sound cues correlated with the content displayed in the VR headset towards the at least one of the given persons associated with the second probability exceeding the threshold.

In some cases, the at least one model is person-specific or one or more of: gender specific, race-specific, or age-range specific.

In some cases, the physiological parameters include at least one, or a combination of two or more of: heart rate, heart rate variability, sweat level, respiration rate, skin color, pupil dilation, blood pressure, muscle tension, body temperature, eye gaze movement, microexpression, body movement, electroencephalography (EEG) readings, voice phonation, voice pitch, voice loudness, or voice rate.

In some cases, the sensors include at least one of: one or more contact sensors, or one or more proximity or remote sensors such as, but not limited to: lasers, radars, cameras, infrared (IR) sensors, Terahertz radiation sensors, ultrasound sensors, or acoustic sensors.

In accordance with a fifth aspect of the presently disclosed subject matter, there is provided a method, comprising: obtaining (a) information of a planned driving route of a driverless autonomous vehicle and (b) an indication of a location of the driverless autonomous vehicle on the planned driving route; and providing one or more instructions to perform one or more motion sickness prevention measures, based on the information and on the indication.

In some cases, the method further comprises performing the motion sickness prevention measures.

In some cases, the providing is also based on past information of motion sickness experienced by one or more passengers riding along the planned driving route.

In some cases, the at least one of the passengers is a current passenger riding in the driverless autonomous vehicle on the planned driving route.

In some cases, the providing is also based on vehicle movement information obtained from one or more sensors monitoring movements of the driverless autonomous vehicle.

In some cases, the motion sickness prevention measures include one or more of: providing a first subliminal or explicit visual cue that is indicative of a global horizon to at least one passenger of the driverless autonomous vehicle; providing a second subliminal or explicit visual cue that is indicative of a predicted next turn of the vehicle along the driving route to the at least one passenger; correlating visual content provided to the at least one passenger to the driving route, giving rise to correlated content; changing the vehicle's windows transparency; displaying the content provided to the at least one passenger in infinity; changing the vehicle's internal lighting strength; changing a temperature within the vehicle; projecting encoring lights to be viewed by the at least one passenger; projecting flashing lights towards the at least one passenger; reducing a sensitivity of the vestibular system of the at least one passenger; influencing the vestibular system of the at least one passenger by transmitting ultrasound waves; eliminating one or more induction vibrations within a frequency range between 240 Hz and 260 Hz that surround at the at least one passenger; inducing vibrations on at least part of the at least one passenger; providing haptic feedback to the at least one passenger; playing one or more stress reducing audio or visual recordings to the at least one passenger; generating an automatic conversation with the at least one passenger; inducing or enhancing pressure on at least part of the at least one passenger; moving a chair of the at least one passenger; generating one or more blasts of wind cues from different positions surrounding the at least one passenger towards the at least one passenger; generating one or more sound cues from different positions surrounding the at least one passenger towards the at least one passenger; stopping the driverless autonomous vehicle; slowing down the driverless autonomous vehicle; limiting the driverless autonomous vehicle's accelerations in at least one axis of movement; calculating a new driving route for the driverless autonomous vehicle; generating one or more blasts of wind cues correlated with the correlated content from different positions surrounding the at least one passenger towards the at least one passenger; or generating one or more sound cues correlated with the correlated content from different positions surrounding the at least one passenger towards the at least one passenger.

In accordance with a sixth aspect of the presently disclosed subject matter, there is provided a method, comprising: obtaining an orientation measurement from at least one orientation measurement sensor of a driverless autonomous vehicle; and displaying content including a subliminal or explicit visual cue that is indicative of a global horizon aligned with the orientation measurement to at least one passenger of the driverless autonomous vehicle.

In accordance with a seventh aspect of the presently disclosed subject matter, there is provided a non-transitory computer readable storage medium having computer readable program code embodied therewith, the computer readable program code, executable by a controller of a computer to perform a method of: (a) providing at least one model indicative of a relationship between first values of one or more physiological parameters of a group of one or more persons and a first probability of the persons within the group to get motion sickness within one or more given time periods; (b) obtaining, from one or more sensors, second values of the physiological parameters, the second values being readings obtained by the sensors from one or more given persons; (c) estimating, based on the second values and on the model, a second probability of each of the given persons to get motion sickness; and (d) providing one or more instructions to perform one or more motion sickness prevention measures for at least one of the given persons upon the second probability of the at least one of the given persons exceeding a threshold to at least reduce the second probability.

In accordance with an eighth aspect of the presently disclosed subject matter, there is provided a non-transitory computer readable storage medium having computer readable program code embodied therewith, the computer readable program code, executable by a controller of a computer to perform a method of: obtaining (a) information of a planned driving route of a driverless autonomous vehicle and (b) an indication of a location of the driverless autonomous vehicle on the planned driving route; and providing one or more instructions to perform one or more motion sickness prevention measures, based on the information and on the indication.

In accordance with a ninth aspect of the presently disclosed subject matter, there is provided a non-transitory computer readable storage medium having computer readable program code embodied therewith, the computer readable program code, executable by a controller of a computer to perform a method of: obtaining an orientation measurement from at least one orientation measurement sensor of a driverless autonomous vehicle; and displaying content including a subliminal or explicit visual cue that is indicative of a global horizon aligned with the orientation measurement to at least one passenger of the driverless autonomous vehicle.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a schematic illustration of an operation of a motion sickness prediction and prevention system, in accordance with the presently disclosed subject matter;

FIG. 2 is a block diagram schematically illustrating one example of a motion sickness prediction system, in accordance with the presently disclosed subject matter;

FIG. 3 is a block diagram schematically illustrating a first example of a motion sickness prevention system, in accordance with the presently disclosed subject matter;

FIG. 4 is a block diagram schematically illustrating a second example of a motion sickness prevention system, in accordance with the presently disclosed subject matter;

FIG. 5 is a block diagram schematically illustrating a third example of a motion sickness prevention system, in accordance with the presently disclosed subject matter;

FIG. 6 is a flowchart illustrating an example of a sequence of operations performed by a motion sickness prediction and prevention system, in accordance with the presently disclosed subject matter;

FIG. 7 is a flowchart illustrating a first example of a sequence of operations performed by a motion sickness prevention system, in accordance with the presently disclosed subject matter; and

FIG. 8 is a flowchart illustrating a second example of a sequence of operations performed by another motion sickness prevention system, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “providing”, “obtaining”, “predicting”, “performing”, “repeating”, “displaying”, “projecting”, “inducing”, “reducing”, “influencing”, “enhancing”, “generating”, “playing”, “stopping”, “slowing down”, “limiting”, “calculating”, “changing”, “moving”, “correlating” or the like, include actions and/or processes, including, inter alia, actions and/or processes of a computer, that manipulate and/or transform data into other data, said data represented as physical quantities, e.g. such as electronic quantities, and/or said data representing the physical objects. The terms “computer”, “processor”, and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

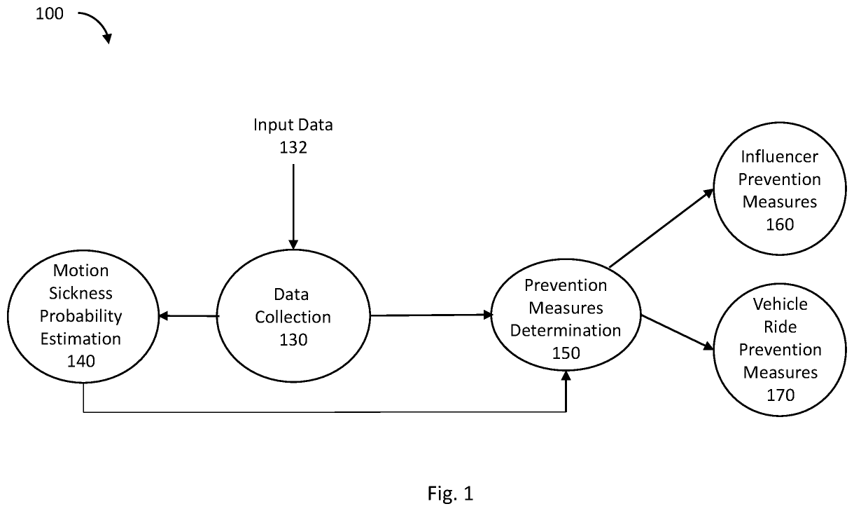

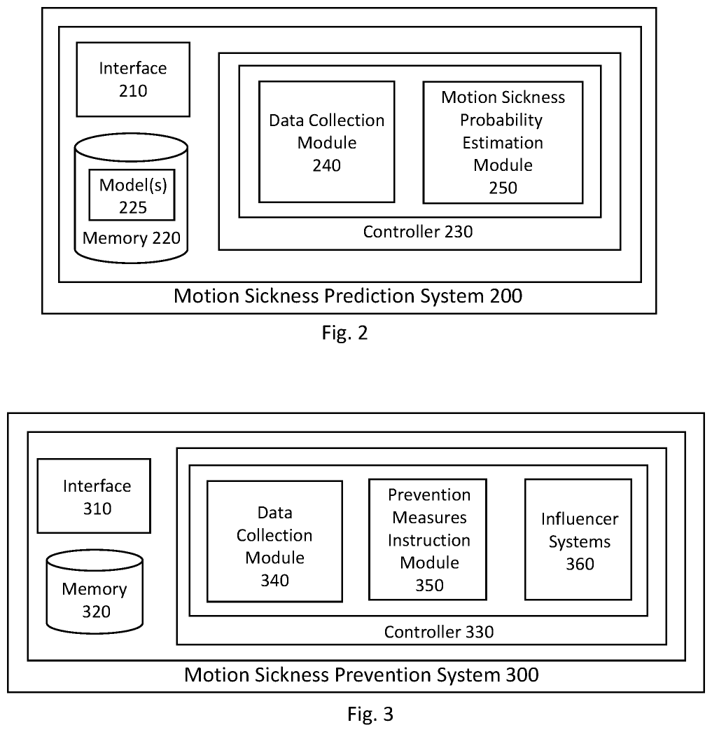

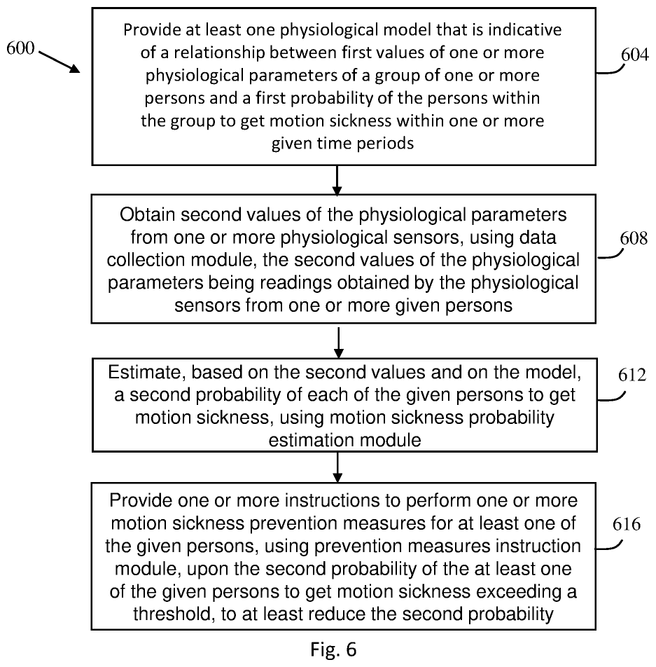

In embodiments of the presently disclosed subject matter, fewer, more and/or different stages than those shown in FIGS. 6-8 may be executed. In embodiments of the presently disclosed subject matter one or more stages illustrated in FIG. 6 may be executed in a different order and/or one or more groups of stages may be executed simultaneously. FIGS. 1-5 illustrate a general schematic of the system architecture in accordance with an embodiment of the presently disclosed subject matter. Each module in FIGS. 2-5 can be made up of any combination of software, hardware and/or firmware that performs the functions as defined and explained herein. The modules in FIGS. 2-5 may be centralized in one location or dispersed over more than one location. In other embodiments of the presently disclosed subject matter, the system may comprise fewer, more, and/or different modules than those shown in FIGS. 2-5.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Bearing this in mind, attention is drawn to FIG. 1, a schematic illustration of an operation 100 of motion sickness prediction and prevention system, in accordance with the presently disclosed subject matter.

In accordance with the presently disclosed subject matter, motion sickness prediction and prevention system can, optionally, include a motion sickness prediction system configured to estimate a probability of each of one or more given persons to get motion sickness prior to an appearance (or explicit declaration/statement) of motion sickness symptoms in the given persons. In some cases, the probability can be estimated continuously.

In addition, motion sickness prediction and prevention system can, optionally, include a motion sickness prevention system to perform one or more motion sickness prevention measures for one or more given persons. In some cases, motion sickness prevention system can be configured to perform one or more motion sickness prevention measures for a given person upon an estimated probability of the given person to get motion sickness exceeding a threshold. Additionally, or alternatively, in some cases, motion sickness prevention system can be configured to perform one or more motion sickness prevention measures for a given person irrespective of an estimated probability of the given person to get motion sickness.

In some cases, the given persons for whom motion sickness prevention measures can be performed are vehicle passengers in a vehicle driving along a driving route. In some cases, the vehicle can be a driverless (autonomous) vehicle, however it is to be noted that whenever reference is made in this description to autonomous vehicles, a human-driven/operated vehicles is also contemplated and the motion sickness prediction and prevention solutions disclosed herein are applicable to such human-driven/operated vehicles, mutatis mutandis. In some cases, the given persons for whom motion sickness prevention measures can be performed are consumers of virtual reality (VR) wearing a VR headset to consume the VR. It is to be noted that all references in the present application to consumption of VR content are also applicable to consumption of Augmented Reality (AR) content or consumption of Mixed Reality (MR) content, and all references in the present application to a VR headset are also applicable to an AR headset, a MR headset, or direct projection of VR, AR or MR content to the eye.

The motion sickness prediction system can be a standalone system or can be integrated with the motion sickness prevention system as part of a motion sickness prediction and prevention system. Moreover, the motion sickness prevention system can be a standalone system or can be integrated with the motion sickness prediction system as part of a motion sickness prediction and prevention system. In addition, the motion sickness prediction and prevention system can be divided into two or more systems in any manner that is desired.

The operation 100 of motion sickness prediction and prevention system can include a data collection 130 process, the data collection 130 process being configured to obtain input data 132. Input data 132 can include, but is not limited to, one or more of the following: physiological data, vehicle passenger behavioral data, vehicle ride data, or customer data.

The physiological data can include values of one or more physiological parameters of one or more given persons. Examples of physiological parameters are provided further herein, inter alia with reference to FIG. 6.

The vehicle passenger behavioral data can include values of one or more vehicle passenger behavioral parameters of one or more given vehicle passengers. Examples of vehicle passenger behavioral parameters are provided further herein, inter alia with reference to FIG. 6.

The vehicle ride data can include values of one or more vehicle ride parameters. In some cases, the vehicle ride data can originate from other vehicles (e.g., information regarding traffic along a driving route, obstacles along a driving route, etc.). Examples of vehicle ride parameters are provided further herein, inter alia with reference to FIGS. 6 to 8.

The customer data can include, for example, at least one of: explicit statements/declarations by one or more persons that they feel that they have motion sickness symptoms, past information of motion sickness experienced by one or more persons, or direct instructions by one or more persons to perform motion sickness prevention measures.

Returning to the operation 100 of motion sickness prediction and prevention system, the operation 100 can, optionally, include motion sickness probability estimation 140, performed by motion sickness prediction system, the motion sickness probability estimation 140 being configured to estimate a probability of each of one or more given persons to get motion sickness, based on input data 132 and on at least one model (not shown), as detailed further herein, inter alia with reference to FIG. 6.

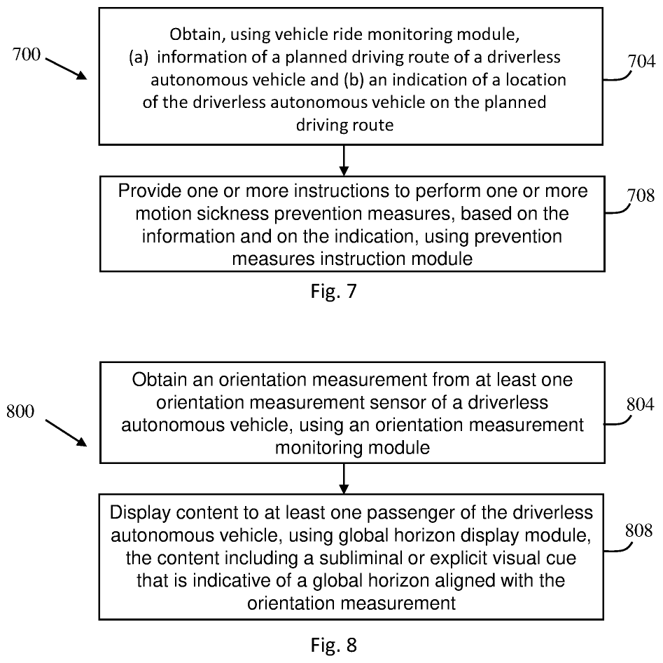

The operation 100 can, optionally, include a prevention measures determination 150 process, performed by motion sickness prevention system, the prevention measures determination 150 process being configured to provide one or more instructions to perform one or more motion sickness prevention measures for at least one given person. The motion sickness prevention measures can be one or more of: influencer prevention measures 160, vehicle ride prevention measures 170, or alerts. In some cases, prevention measures determination 150 process can be configured to provide one or more instructions to one or more influencer systems (not shown) to perform one or more influencer prevention measures 160 for at least one given person, as detailed further herein, inter alia with reference to FIGS. 6 to 8. Examples of influencer systems are provided further herein, inter alia with reference to FIG. 3. Additionally, or alternatively, in some cases, prevention measures determination 150 process can be configured to provide one or more instructions to a vehicle ride computer (not shown) to perform one or more vehicle ride prevention measures 170 for at least one given person, as detailed further herein, inter alia with reference to FIGS. 6 and 7. Additionally, or alternatively, in some cases, prevention measures determination 150 process can be configured to provide at least one instruction to an alert module (not shown) to issue at least one alert.

In some cases, prevention measures determination 150 process can be configured to provide one or more instructions to perform one or more motion sickness prevention measures for at least one given person based on an estimated probability of one or more given persons including the at least one given person to get motion sickness, as detailed further herein, inter alia with reference to FIG. 6, thereby at least reducing the probability of the at least one given person to get motion sickness.

Additionally, or alternatively, in some cases, prevention measures determination 150 process can be configured to provide one or more instructions to perform one or more motion sickness prevention measures for at least one given person irrespective of a probability of the given person to get motion sickness, as detailed further herein, inter alia with reference to FIGS. 7 and 8.

Additionally, or alternatively, in some cases, prevention measures determination 150 process can be configured to provide one or more instructions to perform one or more prevention measures, in response to one or more direct instructions by one or more given persons to perform prevention measures.

Attention is now drawn to FIG. 2, a block diagram schematically illustrating one example of a motion sickness prediction system 200, in accordance with the presently disclosed subject matter.

In accordance with the presently disclosed subject matter, motion sickness prediction system 200 can comprise an interface 210 to enable the motion sickness prediction system 200 to send and receive data. In some cases, the interface 210 can be a network interface that is configured to connect the motion sickness prediction system to a communication network for sending data to and receiving data from one or more computing devices that are connected to the communication network.

Motion sickness prediction system 200 can further comprise or be otherwise associated with memory 220 (e.g. a database, a storage system, a memory including Read Only Memory—ROM, Random Access Memory—RAM, or any other type of memory, etc.), being configured to store data. Memory 220 can be further configured to enable retrieval and/or update and/or deletion of the stored data. It is to be noted that, in some cases, memory 220 can be distributed (e.g., memory 220 can be part of a cloud memory infrastructure accessible via an Internet connection).

Memory 220 can be configured to store one or more models 225. In some cases, the models 225 can include at least one physiological model. Additionally, or alternatively, in some cases, the models 225 can include at least one vehicle ride model. Additionally, or alternatively, in some cases, the models 225 can include at least one vehicle passenger behavior model. Details of the models 225 are provided further herein, inter alia with reference to FIG. 6.

In some cases, one or more of the models 225 can be created or adapted in accordance with statistical methodologies and/or machine-learning techniques. Additionally, or alternatively, in some cases, one or more of the models 225 can be created or adapted manually. In some cases, one or more of the models 225 can be adapted based on parameters associated with the models (i.e., physiological parameters, vehicle ride parameters, and/or vehicle passenger behavioral parameters, as relevant) that are monitored during vehicle movement. In some cases, one or more models 225 can be created or adapted based on explicit statements/declarations by one or more persons that they feel that they have motion sickness symptoms.

Motion sickness prediction system 200 further comprises a controller 230. Controller 230 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant motion sickness prediction system 200 resources and for enabling operations related to motion sickness prediction system 200 resources.

Controller 230 can be configured to include data collection module 240 and motion sickness probability estimation module 250.

Data collection module 240 can be configured, using the data collection 130 process, to obtain input data 132.

Motion sickness probability estimation module 250 can be configured to perform motion sickness probability estimation 140, based on at least some of the input data collected by data collection module 240, to estimate a probability of each of one or more given persons to get motion sickness, as detailed further herein, inter alia with reference to FIG. 6.

Attention is now drawn to FIG. 3, a block diagram schematically illustrating a first example of a motion sickness prevention system 300, in accordance with the presently disclosed subject matter.

In accordance with the presently disclosed subject matter, motion sickness prevention system 300 can comprise an interface 310 to send and receive data. In some cases, the interface 310 can be a network interface that is configured to connect the motion sickness prevention system 300 to a communication network for sending data to and receiving data from one or more computing devices that are connected to the communication network.

Motion sickness prevention system 300 can further comprise or be otherwise associated with memory 320 (e.g. a database, a storage system, a memory including Read Only Memory—ROM, Random Access Memory—RAM, or any other type of memory, etc.), being configured to store data. Memory 320 can be further configured to enable retrieval and/or update and/or deletion of the stored data. It is to be noted that in some cases, memory 320 can be distributed (e.g., memory 320 can be part of a cloud memory infrastructure accessible via an Internet connection).

Motion sickness prevention system 300 further comprises a controller 330. Controller 330 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant motion sickness prevention system 300 resources and for enabling operations related to motion sickness prevention system 300 resources.

Controller 330 can be configured to include, optionally, data collection module 340. Controller 330 can also be configured to include prevention measures instruction module 350 and one or more influencer systems 360.

Data collection module 340 can be configured, using the data collection 130 process, to obtain input data 132.

Prevention measures instruction module 350 can be configured, using prevention measures determination 150 process, to provide one or more instructions to perform one or more motion sickness prevention measures for at least one given person, as detailed earlier herein, inter alia with reference to FIG. 1, and further herein, inter alia with reference to FIGS. 6 to 8.

Influencer systems 360 can be configured to perform one or more influencer prevention measures 160. The influencer systems 360 can include, but are not limited to, at least one of: visual sensory influencer systems, vestibular sensory influencer systems, or somatosensory influencer systems. In some cases, a plurality of influencer systems 360 can be configured to perform a single influencer prevention measure 160. In some cases, the influencer prevention measures 160 can provide cues to a brain of a given person that reduce confusion at the brain when cues provided to the brain by the given person's visual system, his/her vestibular system, and his/her somatosensory system regarding the motion experienced by the given person are not aligned.

Visual Sensory Influencer Systems

Visual sensory influencer systems can be configured to provide a given person with visual cues. These visual cues can then be processed by the brain of the given person, thereby at least reducing a probability of the given person to get motion sickness. In some cases, the given person may have no view or a limited view of the outside world, and the visual cues can compensate for the given person's lack of a view or limited view of the outside world.

The visual sensory influencer systems can include at least one of: a subliminal or explicit indicator system, an adaptive content system, a transparency adjustment system, an infinity content display system, an encoring lights display system, or a flashing lights display system. What follows is a description of these systems, noting that such systems (or some of them) can provide output on various types of displays/screens, infotainment systems and or VR systems, including non-purposely built systems that exist in vehicles.

Subliminal or Explicit Indicator System

A subliminal or explicit indicator system can be configured to display a subliminal or explicit visual cue to a given person when the given person has no view or a limited view of the outside world. In some cases, the given person can be a vehicle passenger riding in a vehicle driving along a driving route, a consumer of VR content via a VR headset, or both.

In some cases, the subliminal or explicit visual cue displayed to the given person can comprise an artificial horizon that is indicative of a global horizon, the artificial horizon being a single straight line of light that accords with an orientation of the given person relative to the Earth's horizon, the artificial horizon being displayed to be detected by a peripheral vision of the given person (just like the real horizon is detected by the peripheral vision). The orientation of the given person relative to the Earth's horizon can be measured by an orientation measurement sensor (for example, a gyroscope, one or more gages, one or more accelerometers, etc.), and the artificial horizon can be generated in accordance with this measurement.

In some cases, in which the given person is a vehicle passenger in a vehicle driving along a driving route, the subliminal or explicit visual cue displayed to the given person can comprise an artificial travel prediction (in addition to, or as an alternative to, the artificial horizon), the artificial travel prediction including two or more curved lines of light that are indicative of a predicted next curve along a planned driving route for the vehicle. In some cases, the artificial travel prediction can be connected to the artificial horizon above the artificial horizon. The planned driving route can be determined, for example, by a computer-based mapping system. The computer-based mapping system can be, for example, a mapping system that is aware of potential driving routes for the vehicle and locations of other vehicles on the potential driving routes.

In some cases, the subliminal or explicit visual cue displayed to the given person can comprise an artificial horizon, whether or not it is supplemented by an artificial travel prediction, and can further comprise lines of light extending outwards both above and below the artificial horizon, the lines of light extending upwards and outwards from the artificial horizon being of a sky blue color and the lines of light extending downwards and outwards from the artificial horizon being of an earth ground brown color.

In some cases, the subliminal or explicit visual cue can be displayed in visual (for example, video) content being viewed by the given person (e.g. on various types of displays/screens, infotainment systems and or VR systems). Additionally, or alternatively, in some cases, the subliminal or explicit visual cue can be displayed on one or more fixed objects that are not displaying visual content (for example, one or more sides of a vehicle in which the given person is a vehicle passenger).

In some cases, a display adjustment system can be configured to adjust over time one or more of the following parameters associated with a display of the subliminal or explicit visual cue to the given person: a rate at which the subliminal or explicit visual cue is displayed, a light intensity at which the subliminal or explicit visual cue is displayed, or the vividness of colors in one or more lines of light that are displayed in the subliminal or explicit visual cue. In some cases, one or more of the aforesaid parameters associated with a display of the subliminal or explicit visual cue to the given person can be increased as an estimated probability of the given person to get motion sickness increases in order to assist the given person in processing the subliminal or explicit visual cue. Additionally, or alternatively, in some cases, one or more of such aforesaid parameters can be decreased as an estimated probability of the given person to get motion sickness decreases.

In some cases, a rate at which the subliminal or explicit visual cue is displayed in video content can vary between a single frame per second to 30 frames per second (i.e., an explicit display of the visual cue). In some cases, the rate at which the subliminal visual cue is displayed in video content can be increased as an estimated probability of the given person to get motion sickness increases. Additionally, or alternatively, in some cases, the rate at which the subliminal visual cue is displayed in video content can be decreased as an estimated probability of the given person to get motion sickness decreases.

It is to be noted that in cases in which the visual cue is explicitly displayed, the light intensity at which the explicit visual cue is displayed and/or the vividness of colors in one or more lines of light that are displayed in the explicit visual cue can be varied. In some cases, the light intensity at which the explicit visual cue is displayed and/or the vividness of colors in one or more lines of light that are displayed in the explicit visual cue can be increased as an estimated probability of the given person to get motion sickness increases. Additionally, or alternatively, in some cases, the light intensity at which the explicit visual cue is displayed and/or the vividness of colors in one or more lines of light that are displayed in the explicit visual cue can be decreased as an estimated probability of the given person to get motion sickness decreases.

Adaptive Content System

An adaptive content system can be configured to correlate visual content (including VR content) viewed by a vehicle passenger in a vehicle driving along a driving route to the driving route, giving rise to correlated content. The visual content can be correlated to a predicted next curve(s) or turn(s) along a planned driving route, or a current curve or turn along a driving route (e.g., video content can be adjusted to move rightward in accordance with a predicted or current right turn of the vehicle). Additionally, or alternatively, in some cases, the visual content can be correlated to a current orientation of the vehicle relative to the Earth's horizon. Determination of the planned driving route and of the current orientation of the vehicle relative to the Earth's horizon can be performed as discussed above with respect to the subliminal indicator system.

Transparency Adjustment System

Transparency adjustment system can be configured to increase a transparency of at least one side of a vehicle and/or a transparency of a VR headset. In some cases, the transparency of the at least one side of the vehicle and/or the transparency of the VR headset can be increased until full transparency is reached (including, for VR, stopping projection of content in the VR headset). As such, the transparency adjustment system can be configured to provide a given person with an enhanced view of the world external to the vehicle and/or the VR headset to better align the cues provided to a given person's brain by his/her visual system with the cues provided to the given person's brain by his/her vestibular system. The given person can be a vehicle passenger in a vehicle driving along a driving route, a VR consumer wearing a VR headset, or both.

Infinity Content Display System

An infinity content display system can be configured to present visual content (for example, a book) to a given person in infinity display or to create one or more infinite cues in visual content that is being viewed by the given person, thereby compensating for a loss of spatial orientation by the given person resulting from a close focus of the eyes of the given person on the visual content. By enhancing an understanding by the given person of his/her spatial orientation, infinity content display system can assist the given person in processing subliminal, explicit, or natural visual cues, thereby at least reducing an estimated probability of the given person to get motion sickness. In some cases, the given person can be a vehicle passenger in a vehicle driving along a driving route, a consumer of VR content wearing a VR headset, or both.

Encoring Lights Display System

Encoring lights display system can be configured to project encoring lights to be viewed by an eye of a given person at a certain point of an eye pupil of the eye. In some cases, the encoring lights can be projected directly on the eye pupil. Alternatively, in some cases, the encoring lights can be projected onto an object to be viewed by the eye pupil. For example, the encoring lights can be projected onto glasses or contact lenses of the given person to be viewed by the eye pupil. The given person can be, for example, a vehicle passenger in a vehicle driving along a driving route, a consumer of VR content wearing a VR headset, or both.

In some cases, encoring lights display system can be configured to vary one or both of the following parameters: a rate at which the encoring lights are projected and an intensity at which the encoring lights are projected. In some cases, one or both of the aforesaid parameters can be increased in accordance with an increase in a probability of the given person to get motion sickness. Additionally, or alternatively, in some cases, one or both of the aforesaid parameters can be decreased in accordance with a decrease in a probability of the given person to get motion sickness.

Flashing Lights Display System

Flashing lights display system can be configured to project one or more flashing (i.e., blinking) lights towards a given person to be viewed by the given person. The given person can be, for example, a vehicle passenger in a vehicle driving along a driving route, a consumer of VR content wearing a VR headset, or both.

In some cases, flashing lights display system can be configured to vary one or both of the following parameters: a rate at which the flashing lights are projected and an intensity at which the flashing lights are projected. In some cases, one or both of the aforesaid parameters can be increased in accordance with an increase in a probability of the given person to get motion sickness. Additionally, or alternatively, in some cases, one or both of the aforesaid parameters can be decreased in accordance with a decrease in a probability of the given person to get motion sickness.

Vestibular Sensory Influencer Systems

Ultrasound Wave Generator

An example of a vestibular sensory influencer system is an ultrasound wave generator. The ultrasound wave generator can be configured to transmit continuous or momentary ultrasound waves into at least one ear (i.e., the vestibular system) of a given person.

In some cases, the ultrasound waves can be transmitted into the ear of the given person to numb an area of the ear, thereby reducing a sensitivity of the vestibular system of the given person. The given person can be, for example, a vehicle passenger in a vehicle driving along a driving route, a consumer of VR content via a VR headset, or both.

In some cases, the ultrasound waves can be transmitted into the ear of the given person to influence the given person's vestibular system. In some cases, the ultrasound waves can be transmitted into the ear of the given person to create movement of ear fluid or crystals (i.e., nerves in the ear) in one or more canals of the ear in a direction counter to a direction or a predicted direction of angular movement of the vehicle (i.e., movement of the vehicle through a curve or turn in a driving route), thereby countering the movement of ear fluid or crystals in the direction or the predicted direction of the angular movement of the vehicle. In such cases, the given person can be a vehicle passenger in a vehicle driving along a driving route, including a vehicle passenger that is viewing VR content.

In some cases, as part of a complex solution for isolating a given person from his/her external environment, the ultrasound wave generator can be configured to transmit ultrasound waves into at least one ear of the given person to numb an area of the ear or to counter the movement of ear fluid or crystals in the ear in a direction or a predicted direction of an angular movement of the vehicle while another mechanism prevents the given person from having a view of the outside world (e.g., by increasing an opaqueness of one or more sides of a vehicle, by generating eye blockage, etc.).

In some cases, the ultrasound waves can be transmitted into the ear of the given person to create movement of ear fluid or crystals in one or more canals of the ear in a direction that aligns with a direction of movement of the visual content to align cues provided to the given person's brain by his/her vestibular system to cues provided to the given person's brain by his/her visual system. In such cases, the given person can be, for example, a vehicle passenger that is viewing visual, including VR, content, or a consumer of VR content.

In some cases, the ultrasound wave generator can contact a body of the given person when the given person is consuming VR. In some cases, the ultrasound wave generator can be located in a chair of the given person when the given person is a vehicle passenger, a consumer of VR, or both. In some cases, the ultrasound wave generator can be located at any location in a vehicle.

Chair/Seat Movement Mechanism

Another example of a vestibular sensory influencer system is a chair/seat movement mechanism. The chair/seat movement mechanism can be configured to move a chair/seat of a given person.

In some cases in which the given person is a vehicle passenger in a vehicle driving along a driving route, the chair/seat movement mechanism can be configured to slowly move the chair/seat of the given person prior to a predicted angular movement of the vehicle in a direction of the predicted angular movement, and to move the chair/seat of the given person during the predicted angular movement in a direction that is counter to the angular movement, thereby reducing one or more accelerations of the vehicle that are sensed by the given person's vestibular system during the predicted angular movement (by spreading out the accelerations over time, the accelerations of the vehicle that are sensed by the vestibular system are reduced). Additionally, in some cases, the chair/seat of the vehicle can also be titled prior to the predicted angular movement in a direction of the predicted angular movement, and the tilting can be reversed during the predicted angular movement.

In some cases in which the given person is a vehicle passenger in a vehicle driving along a driving route, and in which the vehicle begins to perform an angular movement without prior movement of the chair/seat of the vehicle passenger in anticipation of the angular movement (for example, due to the angular movement not being predicted), the chair/seat movement mechanism can be configured, during the angular movement, to tilt the chair/seat in a direction of the angular movement, or alternatively, to move the chair/seat in a direction that is counter to the angular movement, thereby reducing one or more accelerations of the vehicle that are sensed by the given person's vestibular system during the angular movement.

In some cases in which the given person is viewing visual content (whether or not the given person is a vehicle passenger), including VR content, the chair/seat movement mechanism can be configured to move the chair/seat of the given person in a direction of movement of the visual content to support the cue provided by the given person's visual system to his/her brain.

Somatosensory Influencer Systems

The somatosensory influencer systems can be configured to activate sensory receptors of a somatosensory system of a given person. In some cases, the somatosensory influencer systems can be configured to reduce a sensitivity of a somatosensory system of a given person. In some cases, the somatosensory influencer systems can be configured to support or weaken cues provided to a brain of a given person by either the given person's visual system and/or the given person's vestibular system regarding the motion experienced by the given person in cases in which there is a dissonance between cues that are provided to the brain by the given person's visual system and cues provided to the brain by the given person's vestibular system.

The somatosensory influencer systems can include at least one of: an induction vibrations elimination system, a vibrations induction system, a control handle, pressure applying system, a wind blast generation system, or a sound cues generation system. What follows is a description of these systems.

Induction Vibrations Elimination System

Induction vibrations elimination system can be configured to eliminate one or more induction vibrations within a frequency range of 240 Hz to 260 Hz that surround a vehicle passenger in a vehicle, since vibration frequencies in the aforementioned frequency range in a vehicle contribute to bad feelings for the vehicle passengers. The induction vibrations within the aforementioned frequency range can be eliminated passively by using shock absorbers or actively by generating destructive interference.

Vibrations Induction System

Vibrations induction system can be configured to induce vibrations at various frequencies on at least part of a given person to reduce a sensitivity of a somatosensory system of the given person. The given person can be, for example, a vehicle passenger driving in a vehicle along a driving route, a consumer of VR via a VR headset, or both.

Control Handle

A control handle can be made available to a given person to return feelings of control, spatial orientation, and feelings of movements to the given person, thereby reducing a sensitivity of a somatosensory system of the given person. The control handle can be a passive control handle that reduces a sensitivity of a somatosensory system of a vehicle passenger when the vehicle passenger grips the control handle, in accordance with the law of action and reaction. The control handle can also be an active control handle. In some cases, the active control handle can provide haptic feedback to a vehicle passenger via vibrations or small movements of the handle in a predicted direction and/or direction of the vehicle movement. In some cases, the active control handle can provide haptic feedback to a vehicle passenger that is viewing content, including VR content, or to a consumer of VR content by inducing vibrations or small movements of the handle in a direction of movement of the content.

Pressure Applying System

A pressure applying system (for example, airbags) that is present in a chair or a seat of a given person can be configured to apply pressure at different parts of the person's body.

In some cases in which the given person is a vehicle passenger in a vehicle driving along a driving route, the pressure applying system can be configured to apply pressure on the given person to provide cues to the given person in anticipation of a predicted angular movement of the vehicle prior to the predicted angular movement. In some cases, the pressure applying system can be configured to apply pressure on the given person to communicate to the given person that a predicted angular movement will occur prior to the occurrence thereof. For example, prior to a predicted right turn of the vehicle, the pressure applying system can be configured to communicate to the given person that a right turn is to be performed by applying light pressure to the right side of the given person.

Alternatively, in some cases, the pressure applying system can be configured to apply pressure on the given person prior to a predicted angular movement to provide cues to the given person's brain that align with the cues provided to the given person's brain by the vestibular system of the given person when the vehicle begins performing the predicted angular movement. For example, prior to a predicted right turn of the vehicle, the pressure applying system can be configured to apply pressure to the left side of the given person.

In some cases in which the given person is a vehicle passenger in a vehicle driving along a driving route, the pressure applying system can be configured to apply pressure on the given person to support the cues provided by the vestibular system of the given person to his/her brain during an angular movement of the vehicle. For example, during a right turn of the vehicle, the pressure applying system can be configured to apply pressure to the left side of the given person. Alternatively, in some cases, the pressure applying system can be configured to apply pressure on the given person to weaken the cues provided by the vestibular system of the given person to his/her brain during an angular movement of the vehicle. For example, during a right turn of the vehicle, the pressure applying system can be configured to apply pressure to the right side of the given person.

In some cases in which the given person is viewing visual content (whether or not the given person is a vehicle passenger), including VR content, the pressure applying system can be configured to apply pressure on the given person to support the cues provided by the given person's visual system to his/her brain. For example, when the visual content is moving to the right, the pressure applying system can be configured to apply pressure to the left side of the given person.

In some cases, the frequency at which the pressure applying system applies pressure on the given person and/or the speed at which the pressure applying system applies pressure on the given person can be varied. For example, the frequency and/or speed at which the pressure is applied can be increased in accordance with an increase in a probability of a given person to get motion sickness, and/or decreased in accordance with a decrease in a probability of a given person to get motion sickness.

Wind Blast Generation System

Wind blast generation system can be configured to generate one or more blasts of wind cues from different positions surrounding (in 360 degrees of direction) a given person towards the given person.

In some cases, the wind blast generation system can be configured to generate one or more blasts of wind cues toward a given person's face at an appropriate temperature to provide the given person with a good feeling.

In some cases in which the given person is a vehicle passenger in a vehicle driving along a driving route, the wind blast generation system can be configured to generate one or more blasts of wind cues towards the vehicle passenger from one or more positions surrounding the vehicle passenger in accordance with a direction of movement of the vehicle or a predicted direction of movement of the vehicle. For example, when a vehicle is moving in a straight line, the wind blast generation system can be configured to generate one or more blasts of wind cues from a position in front of the vehicle passenger. As an additional example, when a vehicle is turning right, the wind blast generation system can be configured to generate one or more blasts of wind cues from a position to the right of the vehicle passenger during the turn, and, in some cases, prior to the turn (as a means of communicating to the vehicle passenger that a turn is approaching).

Alternatively, in some cases in which the given person is a vehicle passenger in a vehicle driving along a driving route, the wind blast generation system can be configured to generate one or more blasts of wind cues towards the vehicle passenger to counter a cue provided by the vestibular system of the vehicle passenger regarding movement of the vehicle.

In some cases in which the given person is viewing visual content (whether or not the given person is a vehicle passenger), including VR content, the wind blast generation system can be configured to generate one or more blasts of wind cues towards the given position in accordance with a direction of movement of the visual content to support the cue provided by the given person's visual system to his/her brain. For example, when the visual content is moving rightward, the wind blast generation system can be configured to generate one or more blasts of wind cues from a position to the right of the given person.

In some cases, adaptive content system can be configured to correlate the visual content to a predicted and/or current angular movement of the vehicle, and the wind blast generation system can be configured to generate one or more blasts of wind cues towards the given person from a direction that is correlated to a direction of the correlated content, i.e., the direction of the predicted and/or current angular movement of the vehicle.

In some cases, the velocity of the blasts of wind cues can be varied. For example, the velocity of the blasts of wind cues can be increased in accordance with an increase in a probability of a given person to get motion sickness, and/or decreased in accordance with a decrease in a probability of a given person to get motion sickness.

Sound Cues Generation System

Sound cues generation system can be configured to generate one or more sound cues from different positions surrounding (in 360 degrees of direction) a given person towards the given person.

In some cases in which the given person is a vehicle passenger in a vehicle driving along a driving route, the sound cues generation system can be configured to generate one or more sound cues towards the vehicle passenger from one or more positions surrounding the vehicle passenger in accordance with a direction of movement of the vehicle or a predicted direction of movement of the vehicle. For example, when a vehicle is moving in a straight line, the sound cues generation system can be configured to generate one or more sound cues from a position in front of the vehicle passenger. As an additional example, when a vehicle is turning right, the sound cues generation system can be configured to generate one or more sound cues from a position to the right of the vehicle passenger during the turn, and, in some cases, prior to the turn (as a means of communicating to the vehicle passenger that a turn is approaching).

Alternatively, in some cases in which the given person is a vehicle passenger in a vehicle driving along a driving route, the sound cues generation system can be configured to generate one or more sound cues towards the vehicle passenger to counter a cue provided by the vestibular system of the vehicle passenger regarding movement of the vehicle.

In some cases in which the given person is viewing visual content (whether or not the given person is a vehicle passenger), including VR content, the sound cues generation system can be configured to generate one or more sound cues towards the given person in accordance with a direction of movement of the visual content to support the cue provided by the given person's visual system to his/her brain. For example, when the visual content is moving rightward, the sound cues generation system can be configured to generate one or more sound cues from a position to the right of the given person.

In some cases, adaptive content system can be configured to correlate the direction of movement of the visual content to a predicted and/or current angular movement of the vehicle, and the sound cues generation system can be configured to generate one or more sound cues towards the given person from a direction that is correlated to the direction of movement of the correlated content, i.e., the direction of the predicted and/or current angular movement of the vehicle.

In some cases, one or both of the intensity and frequency of the sound cues can be varied. For example, the intensity and/or frequency of the sound cues can be increased in accordance with an increase in a probability of a given person to get motion sickness, and/or decreased in accordance with a decrease in a probability of a given person to get motion sickness.

Additional Influencer Prevention Systems

Audio or Visual Recordings System

An audio or visual recordings system can play stress reducing audio/speech/music and/or visual recordings. In some cases, the stress reducing audio/speech/music and/or visual recordings can include suggestive messaging.

Automatic Conversation Generation System

Automatic conversation generation system can be configured to initiate an automatic conversation with a given person. In some cases, the automatic conversation can include suggestive messaging. In some cases, the automatic conversation can include instructions for the given person to reduce a probability that the given person will get motion sickness.

Temperature Regulating Device

A temperature regulating device (e.g., a thermostat) can be configured to change a temperature within a room/space in which VR is being consumed, or to change a temperature within a vehicle.

VR Headset Straps

Straps of a VR headset can be configured to provide haptic feedback to consumers of VR content via the VR headset, using any number of means, including vibration and pressure. In some cases, the haptic feedback provided by the straps of the VR headset can be varied. For example, the haptic feedback can be increased in accordance with an increase in a probability of the given person to get motion sickness, and/or decreased in accordance with a decrease in a probability of the given person to get motion sickness.

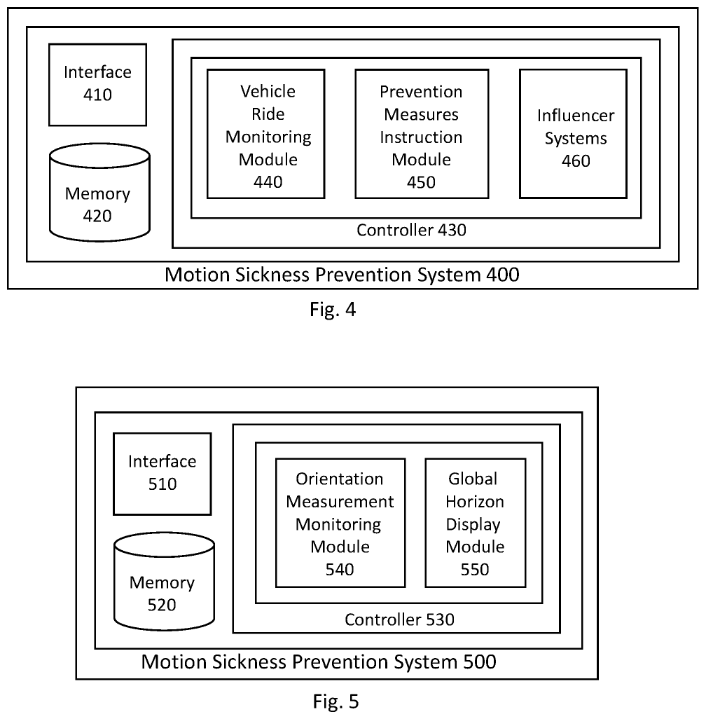

Attention is now drawn to FIG. 4, a block diagram schematically illustrating a second example of a motion sickness prevention system 400, in accordance with the presently disclosed subject matter. Motion sickness prevention system 400 can be configured to prevent one or more vehicle passengers in a driverless (autonomous) vehicle from getting motion sickness.

In accordance with the presently disclosed subject matter, motion sickness prevention system 400 can comprise an interface 410 that is configured to enable motion sickness prevention system 400 to send and receive data. In some cases, the interface 410 can be a network interface that is configured to connect the motion sickness prevention system 400 to a communication network for sending data to and receiving data from one or more computing devices that are connected to the communication network.

Motion sickness prevention system 400 can further comprise or be otherwise associated with memory 420 (e.g. a database, a storage system, a memory including Read Only Memory—ROM, Random Access Memory—RAM, or any other type of memory, etc.), being configured to store data. Memory 420 can be further configured to enable retrieval and/or update and/or deletion of the stored data. It is to be noted that in some cases, memory 420 can be distributed (e.g., memory 420 can be part of a cloud memory infrastructure accessible via an Internet connection).

Motion sickness prevention system 400 further comprises a controller 430. Controller 430 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant motion sickness prevention system 400 resources and for enabling operations related to motion sickness prevention system 400 resources.

Controller 430 can be configured to include vehicle ride monitoring module 440, prevention measures instruction module 450, and one or more influencer systems 460.

Vehicle ride monitoring module 440 can be configured to obtain input data 132, including vehicle ride data associated with a driverless (autonomous) vehicle (or with a human-driven/operated vehicle) that is driving along a planned driving route, and, optionally, customer data associated with the planned driving route, as detailed further herein, inter alia with reference to FIG. 7.

Prevention measures instruction module 450 can be configured to provide one or more instructions to perform one or more motion sickness prevention measures, based on the input data 132 obtained by vehicle ride monitoring module 440, as detailed further herein, inter alia with reference to FIG. 7.

Influencer systems 460 can be configured to perform one or more influencer prevention measures 160, in accordance with instructions received from prevention measures instruction module 450.

Attention is now drawn to FIG. 5, a block diagram schematically illustrating a third example of a motion sickness prevention system 500, in accordance with the presently disclosed subject matter. Motion sickness prevention system 500 can be configured to prevent one or more vehicle passengers in a driverless (autonomous) vehicle from getting motion sickness.

In accordance with the presently disclosed subject matter, motion sickness prevention system 500 can comprise an interface 510 that is configured to enable the motion sickness prevention system 500 to receive and send data. In some cases, the interface 510 can be a network interface that can be configured to connect the motion sickness prevention system 500 to a communication network for sending data to and receiving data from computing devices connected to the communication network.

Motion sickness prevention system 500 can further comprise or be otherwise associated with memory 520 (e.g. a database, a storage system, a memory including Read Only Memory—ROM, Random Access Memory—RAM, or any other type of memory, etc.), being configured to store data. Memory 520 can be further configured to enable retrieval and/or update and/or deletion of the stored data. It is to be noted that in some cases, memory 520 can be distributed (e.g., memory 520 can be part of a cloud memory infrastructure accessible via an Internet connection).

Motion sickness prevention system 500 further comprises a controller 530. Controller 530 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant motion sickness prevention system 500 resources and for enabling operations related to motion sickness prevention system 500 resources.

Controller 530 can be configured to include orientation measurement monitoring module 540 and global horizon display module 550.

Orientation measurement monitoring module 540 can be configured to obtain an orientation measurement from at least one orientation measurement sensor of a driverless (autonomous) vehicle (or of a human-driven/operated vehicle) (e.g., one or more gages, one or more accelerometers, gyroscope).

Global horizon display module 550 can be configured, using the subliminal or explicit indicator system, to display content to at least one passenger in the driverless (autonomous) vehicle, the content including a subliminal or explicit visual cue indicative at least of a global horizon, the indication of the global horizon being aligned with the orientation measurement, as discussed earlier herein, inter alia with reference to FIG. 3.

It is to be noted that, in some cases, motion sickness prediction system 200 and motion sickness prevention systems 300, 400 and 500 can be a single system. It is also to be noted that motion sickness prediction and prevention system can be divided into multiple systems in any manner that is desired.

Attention is now drawn to FIG. 6, a flowchart illustrating an example of a sequence of operations 600 performed by a motion sickness prediction and prevention system, in accordance with the presently disclosed subject matter.

Motion sickness prediction and prevention system can be configured to provide at least one model, the at least one model being one or more of: at least one physiological model, at least one vehicle ride model, or at least one vehicle passenger behavior model.

In some cases, as in the illustrated example, at least one physiological model can be provided, the physiological model being indicative of a relationship between first values of one or more physiological parameters of a group of one or more persons and a first probability of the persons within the group to get motion sickness within one or more given time periods (step 604).

In some cases, the at least one physiological model can include one or more person-specific physiological models. Additionally, or alternatively, in some cases, the at least one physiological model can include one or more group-specific physiological models. Three non-limiting examples of group-specific physiological models are: (a) a physiological model associated with members of a specific gender; (b) a physiological model associated with members of a specific race; and (c) a physiological model associated with members of a specific age range.

Motion sickness prediction and prevention system can be configured to obtain second values of the physiological parameters from one or more physiological sensors (whether contact sensors that require direct contact with the passenger's body, or contactless sensors that do not require contact with the passenger's body), using data collection module 240, the second values of the physiological parameters being readings obtained by the physiological sensors from one or more given persons (step 608).

The physiological parameters can be configured to include, but are not limited to, at least one, or a combination of two or more of: heart rate, heart rate variability, sweat level (obtained using a galvanic skin response (GSR) sensor), respiration rate, skin color, pupil dilation, blood pressure, muscle tension, body temperature, eye gaze movement, pupil behavior, face colorization (blush), microexpression, body movement, electroencephalography (EEG) readings, voice phonation, voice pitch, voice loudness, or voice rate.

The physiological sensors can include, but are not limited to, at least one of: one or more direct contact sensors, or one or more proximity or remote sensors such as, but not limited to: lasers, radars, cameras, infrared (IR) sensors, Terahertz radiation sensors, ultrasound sensors, galvanic skin response (GSR) sensors, or acoustic sensors. A given sensor of the sensors can be configured to obtain values of: (a) a single physiological parameter, or (b) two or more physiological parameters.

Returning to the illustrated example, motion sickness prediction and prevention system can be configured to estimate, based on the second values of the physiological parameters and the at least one physiological model, a second probability of each of the given persons to get motion sickness, using motion sickness probability estimation module 250 (step 612).

In some cases in which the given persons are vehicle passengers in a vehicle driving along a driving route, motion sickness probability estimation module 250 can be configured to estimate the second probability of at least some of the given persons to get motion sickness based on the second values of the physiological sensors, the at least one physiological model, and values of vehicle ride parameters. These vehicle ride parameters can include, but are not limited to, at least one of: a driving route of the vehicle, driving conditions along the driving route of the vehicle (e.g., speed bumps, traffic volume, obstacles on the road, frequency and angle of turns, etc.), a current speed of the vehicle, a predicted speed of the vehicle, a temperature within the vehicle, a type of vehicle and/or specifications for the vehicle (e.g., a car, a truck, the type of truck, horsepower, other specifications for the vehicle), or information of accelerations/decelerations of the vehicle along one or more axes. In some cases, at least some of the values of the vehicle ride parameters can originate from other vehicles (e.g., a given vehicle identifies an obstacle on the road—it provides information of its location to other vehicles).

Motion sickness prediction and prevention system can also be configured to provide one or more instructions to perform one or more motion sickness prevention measures for at least one of the given persons, using prevention measures instruction module 350, upon the second probability of the at least one of the given persons to get motion sickness exceeding a threshold, to at least reduce the second probability (step 616). In some cases, the motion sickness prevention measures to be performed for the at least one of the given persons can be selected based on the results of motion sickness prevention measures previously performed for the at least one of the given persons.

The motion sickness prevention measures can be performed in response to the instructions to perform the motion sickness prevention measures.

In some cases, at least one vehicle ride model can be provided by motion sickness prediction and prevention system. The vehicle ride model is indicative of a relationship between first values of one or more vehicle ride parameters associated with a group of one or more persons and a first probability of the persons within the group to get motion sickness within one or more given time periods.

The vehicle ride parameters can be configured to include, but are not limited to, at least one, or a combination of two or more of: vehicle velocity along a straight axis, vehicle acceleration/deceleration along a straight axis, vehicle acceleration/deceleration along a vertical axis, vehicle acceleration/deceleration along a lateral axis, vehicle velocity along a straight axis at one or more predefined distances prior to a turn, vehicle acceleration/deceleration along a straight axis at one or more predefined distances prior to a turn, a number of turns to be performed by the vehicle within a predetermined time period, vehicle velocity during a given turn, or a position of a vehicle passenger in the vehicle.

In some cases, the at least one vehicle ride model can include one or more vehicle ride models that are specific to a given person. Additionally, or alternatively, in some cases, the at least one vehicle ride model can include one or more group-specific vehicle ride models that are associated with a predefined group of persons. Three non-limiting examples of group-specific vehicle ride models are: (a) a vehicle ride model associated with members of a specific gender; (b) a vehicle ride model associated with members of a specific race; and (c) a vehicle ride model associated with members of a specific age range.

Motion sickness prediction and prevention system can be configured to obtain second values of the vehicle ride parameters that are included in the at least one vehicle ride model from a vehicle (e.g., a vehicle ride computer of the vehicle), using data collection module 240, the second values being used to estimate a second probability of one or more given vehicle passengers in the vehicle to get motion sickness.

Motion sickness prediction and prevention system can be further configured to estimate a second probability of each of the aforesaid given vehicle passengers to get motion sickness, using motion sickness probability estimation module 250, based on the second values of the vehicle ride parameters, the at least one vehicle ride model, and, optionally, second values of other parameters that are provided in the present application, and, optionally, at least one additional model that is provided in the present application.

Motion sickness prediction and prevention system can also be configured to provide one or more instructions to perform one or more motion sickness prevention measures for at least one of the given persons, using prevention measures instruction module 350, upon the second probability of the at least one of the given persons exceeding a threshold, to at least reduce the second probability.

In some cases, the instructions can include instructions (for example, to the vehicle ride computer) to alter “ride envelope” parameters in a limited “ride envelope” mode, the “ride envelope” parameters defining a maximum speed of the vehicle, and/or a maximum acceleration/deceleration of the vehicle in one or more axes of movement. It is to be noted, however, that the instructions to perform motion sickness prevention measures can include instructions to perform any of the influencer measures and/or any other motion sickness prevention measures that are detailed in the present application.

In some cases, the motion sickness prevention measures to be performed for the at least one of the given persons associated with the second probability exceeding the threshold can be selected based on the results of motion sickness prevention measures previously performed for the at least one of the given persons.

The motion sickness prevention measures can be performed in response to the instructions to perform the motion sickness prevention measures.

In some cases in which the given persons are vehicle passengers in a vehicle driving along a driving route, at least one vehicle passenger behavior model can be provided by motion sickness prediction and prevention system. The vehicle passenger behavior model is indicative of a relationship between first values of one or more vehicle passenger behavioral parameters of a group of one or more persons and a first probability of the persons within the group to get motion sickness within one or more given time periods.

The vehicle passenger behavioral parameters can be configured to include, but are not limited to, at least one, or a combination of two or more of:
(a) parameters associated with physical posture, including, but not limited to, at least one, or a combination of two or more of: standing, sitting upright, leaning forward, leaning backward without support of a chair/seat, learning backward in a chair/seat, laying down, crouching, turning (e.g., torso and legs pointing forward and upper body turned in a different direction), or head turning (i.e., body in one direction, head alone in another direction);
(b) parameters associated with counter-ride direction positioning, including, but not limited to, at least one, or a combination of two or more of: a vehicle passenger is seated in a direction that is at an angle of n degrees to a direction of vehicle movement in a clockwise direction, wherein n can be any value between zero degrees and 360 degrees; or a vehicle passenger is seated in a direction of vehicle movement and a head of the vehicle passenger is facing a direction that is an angle of x degrees to the direction of the vehicle movement, wherein x can be any value between −90 degrees and 90 degrees;
(c) parameters associated with voluntary sensory blockage, including, but not limited to, at least one, or a combination of two or more of: eyes of a vehicle passenger being closed; eyes of a vehicle passenger being semi-closed; nose of a vehicle passenger being blocked; mouth of a vehicle passenger being closed; a vehicle passenger being attuned to sound or music from a headset covering the ears of the vehicle passenger, the headset not being a VR headset (or an AR or MR headset); or a vehicle passenger being attuned to VR content from a VR headset covering the ears of the vehicle passenger during movement of the vehicle;
(d) parameters associated with speech of a vehicle passenger, including, but not limited to, at least one, or both of: speech of a vehicle passenger (e.g., a conversation being carried out by the vehicle passenger) into a phone or other computerized system during movement of the vehicle; or conversation of the vehicle passenger with one or more other vehicle passengers in the vehicle during movement of the vehicle;
(e) parameters associated with audio content that is being heard by the vehicle passenger or visual content being seen by the vehicle passenger, including, but not limited to, at least one, or a combination of two or more of: reading, viewing a movie, gaming, or listening to music;
(f) parameters associated with other behavior of the vehicle passenger, including, but not limited to, at least one, or both of: eating or drinking; or
(g) engagement of a vehicle passenger in a physiological immersive or quasi-immersive activity (initiated by the vehicle passenger or part of a prescribed service).

In some cases, the at least one vehicle passenger behavior model can include one or more vehicle passenger behavior models that are specific to a given person. Additionally, or alternatively, in some cases, the at least one vehicle passenger behavior model can include one or more group-specific vehicle passenger behavior models that are associated with a predefined group of persons. Three non-limiting examples of group-specific vehicle passenger behavior models are: (a) a vehicle passenger behavior model associated with members of a specific gender; (b) a vehicle passenger behavior model associated with members of a specific race; and (c) a vehicle passenger behavior model associated with members of a specific age range.

Motion sickness prediction and prevention system can be configured to obtain second values of the vehicle passenger behavioral parameters associated with one or more given persons from one or more vehicle passenger behavioral sensors (e.g. a camera and image analysis, pressure sensors on a chair/seat of the vehicle passenger, remote sensors, Terahertz radiation sensors, lasers, eye movement sensors, radar sensors, voice analysis sensors, etc.), using data collection module 240.

Motion sickness prediction and prevention system can be further configured to estimate a second probability of each of the given persons to get motion sickness, using motion sickness probability estimation module 250, based on the second values of the vehicle passenger behavioral parameters, the at least one vehicle passenger behavior model, and, optionally, second values of other parameters that are provided in the present application, and, optionally, at least one additional model that is provided in the present application.

Motion sickness prediction and prevention system can also be configured to provide one or more instructions to perform one or more motion sickness prevention measures for at least one of the given persons, using prevention measures instruction module 350, upon the second probability of the at least one of the given persons exceeding a threshold, to at least reduce the second probability. In some cases, the motion sickness prevention measures to be performed for the at least one of the given persons can be selected based on the results of motion sickness prevention measures previously performed for the at least one of the given persons.

The motion sickness prevention measures can be performed in response to the instructions to perform the motion sickness prevention measures.

In some cases, the steps of obtaining second values of parameters associated with one or more given persons, using data collection module 240, estimating a second probability of each of the given persons to get motion sickness, using motion sickness probability estimation module 250, and providing one or more instructions to perform one or more motion sickness prevention measures upon the second probability of at least one of the given persons exceeding a threshold, using prevention measures instruction module 350, can be repeated a plurality of times over a time period.

The motion sickness prevention measures can be configured to include one or more of: providing a first subliminal or explicit visual cue that is indicative of at least a global horizon to the at least one of the given persons associated with the second probability exceeding the threshold, using subliminal or explicit indicator system; displaying the content provided to the at least one of the given persons associated with the second probability exceeding the threshold in infinity, using infinity content display system; projecting encoring lights to be viewed by the at least one of the given persons associated with the second probability exceeding the threshold, using encoring lights display system; projecting flashing lights towards the at least one of the given persons associated with the second probability exceeding the threshold, using flashing lights display system; reducing a sensitivity of a vestibular system of the at least one of the given persons associated with the second probability exceeding the threshold, using ultrasound wave generator; influencing the vestibular system of the at least one of the given persons associated with the second probability exceeding the threshold by transmitting ultrasound waves, using ultrasound wave generator; inducing vibrations on at least part of the at least one of the given persons associated with the second probability exceeding the threshold, using vibrations induction system; providing haptic feedback to the at least one of the given persons associated with the second probability exceeding the threshold, using a control handle; playing stress reducing audio or visual recordings for the at least one of the given persons associated with the second probability exceeding the threshold, using audio or visual recordings system; generating an automatic conversation with the at least one of the given persons associated with the second probability exceeding the threshold, using automatic conversation generation system; inducing or enhancing pressure on at least part of the at least one of the given persons associated with the second probability exceeding the threshold, using pressure applying system; moving a chair or seat of the at least one of the given persons associated with the second probability exceeding the threshold, using chair/seat moving mechanism; generating one or more blasts of wind cues from different positions surrounding the at least one of the given persons associated with the second probability exceeding the threshold towards the at least one of the given persons, using wind blast generation system; or generating one or more sound cues from different positions surrounding the at least one of the given persons associated with the second probability exceeding the threshold towards the at least one of the given persons, using sound cues generation system.

In some cases, the first subliminal or explicit visual cue can be provided at a varying display rate, based on the second probability.

In cases in which the given persons are vehicle passengers in a vehicle driving along a driving route, the motion sickness prevention measures can be configured to also include one or more of: stopping the vehicle; slowing down the vehicle; limiting the vehicle's accelerations in at least one axis of movement; limiting the vehicle's speed; calculating a new driving route for the vehicle; providing a second subliminal or explicit indication of a predicted next turn of the vehicle along the driving route to the at least one of the given persons associated with the second probability exceeding the threshold, using subliminal or explicit indicator system; correlating visual content provided to the at least one of the given persons associated with the second probability exceeding the threshold to the driving route, giving rise to correlated content, using adaptive content system; changing the vehicle's windows transparency, using transparency adjustment system; changing the vehicle's internal lighting strength, using display adjustment system; changing a temperature within the vehicle, using temperature regulating device; eliminating one or more induction vibrations within a frequency range of 240 Hz to 260 Hz that surround the at least one of the given persons associated with the second probability exceeding the threshold, using induction vibrations elimination system; generating one or more blasts of wind cues correlated with the correlated content towards the at least one of the given persons associated with the second probability exceeding the threshold, using wind blast generation system; or generating one or more sound cues correlated with the correlated content towards the at least one of the given persons associated with the second probability exceeding the threshold, using sound cues generation system.

In cases in which the given persons are consumers of VR wearing a VR headset, the motion sickness prevention measures can also be configured to include one or more of: stopping projection of content in the VR headset worn by the at least one of the given persons associated with the second probability exceeding the threshold; changing a temperature within a room in which the at least one of the given persons associated with the second probability exceeding the threshold is located, using temperature regulating device; providing haptic feedback to the at least one of the given persons associated with the second probability exceeding the threshold, using straps of the VR headset; changing a transparency of the VR headset, using transparency adjustment system; changing the light intensity of the VR headset, using display adjustment system; moving a chair of the at least one of the given persons associated with the second probability exceeding the threshold to at least partially mimic movements made within the content displayed in the VR headset, using chair/seat movement mechanism; generating one or more blasts of wind cues correlated with the content displayed in the VR headset towards the at least one of the given persons associated with the second probability exceeding the threshold, using wind blast generation system; or generating one or more sound cues correlated with the content displayed in the VR headset towards the at least one of the given persons associated with the second probability exceeding the threshold, using sound cues generation system.

Attention is now drawn to FIG. 7, a flowchart illustrating a first example of a sequence of operations performed by motion sickness prevention system 700, in accordance with the presently disclosed subject matter.

In the illustrated example, motion sickness prevention system 400 can be configured, using vehicle ride monitoring module 440, to obtain the following vehicle ride parameters: (a) information of a planned driving route of a driverless autonomous vehicle and (b) an indication of a location of the driverless autonomous vehicle on the planned driving route (step 704).

Additionally, in some cases, motion sickness prevention system 400 can be configured, using vehicle ride monitoring module 440, to also obtain one or more of: past information of motion sickness experienced by one or more passengers riding along the planned driving route, wherein at least one of the passengers can be a current passenger in the driverless autonomous vehicle; or vehicle movement information obtained from one or more sensors monitoring movements of the driverless autonomous vehicle, the vehicle movement information including a current speed of the vehicle and/or information of accelerations of the vehicle in at least one axis.

Motion sickness prevention system 400 can be further configured to provide one or more instructions to perform one or more motion sickness prevention measures, based on the information and on the indication, using prevention measures instruction module 450 (step 708).

The motion sickness prevention measures can be performed in response to the instructions to perform the motion sickness prevention measures. In some cases, the motion sickness prevention measures to be performed can be selected based on the results of motion sickness prevention measures previously performed for at least one of the passengers in the driverless autonomous vehicle.

The motion sickness prevention measures can be configured to include one or more of: providing a first subliminal or explicit visual cue that is indicative of a global horizon to at least one passenger of the driverless autonomous vehicle, using subliminal or explicit indicator system; providing a second subliminal or explicit visual cue that is indicative of a predicted next turn of the vehicle along the driving route to the at least one passenger, using subliminal or explicit indicator system; correlating visual content provided to the at least one passenger to the driving route, giving rise to correlated content, using adaptive content system; changing the vehicle's windows transparency, using transparency adjustment system; displaying the content provided to the at least one passenger in infinity, using infinity content display system; changing the driverless autonomous vehicle's internal lighting strength, using display adjustment system; changing a temperature within the driverless autonomous vehicle, using temperature regulating device; projecting encoring lights to be viewed by the at least one passenger, using encoring lights display system; projecting flashing lights towards the at least one passenger, using flashing lights display system; reducing a sensitivity of a vestibular system of the at least one passenger, using ultrasound wave generator; influencing the vestibular system of the at least one passenger by transmitting ultrasound waves, using ultrasound wave generator; eliminating one or more induction vibrations within a frequency range of 240 Hz to 260 Hz that surround the at least one passenger, using induction vibrations elimination system; inducing vibrations on at least part of the at least one passenger, using vibrations induction system; providing haptic feedback to the at least one passenger, using a control handle; playing one or more stress reducing audio or visual recordings in a direction of the at least one passenger, using audio or visual recordings system; initiating an automatic conversation with the at least one passenger, using automatic conversation generation system; inducing or enhancing pressure on at least part of the at least one passenger, using pressure applying system; moving a chair of the at least one passenger, using chair/seat moving mechanism; generating one or more blasts of wind cues from different positions surrounding the at least one passenger towards the at least one passenger, using wind blast generation system; generating one or more sound cues from different positions surrounding the at least one passenger towards the at least one passenger, using sound cues generation system; stopping the driverless autonomous vehicle; slowing down the driverless autonomous vehicle; limiting the driverless autonomous vehicle's accelerations in at least one axis of movement; limiting the driverless autonomous vehicle's speed; calculating a new driving route for the vehicle; generating one or more blasts of wind cues correlated with the correlated content from different positions surrounding the at least one passenger towards the at least one passenger, using wind blast generation system; or generating one or more sound cues correlated with the correlated content from different positions surrounding the at least one passenger towards the at least one passenger, using sound cues generation system.

Attention is now drawn to FIG. 8, a flowchart illustrating a second example of a sequence of operations performed for another motion sickness prevention system 500, in accordance with the presently disclosed subject matter.

In the illustrated example, motion sickness prevention system 500 can be configured to obtain an orientation measurement from at least one orientation measurement sensor of a driverless autonomous vehicle, using an orientation measurement monitoring module 540 (step 804).

Motion sickness prevention system 500 can be further configured to display content to at least one passenger of the driverless autonomous vehicle, using global horizon display module 550, the content including a subliminal or explicit visual cue that is indicative of a global horizon aligned with the orientation measurement (step 808).

It is to be noted that, with reference to FIGS. 6 to 8, some of the blocks can be integrated into a consolidated block or can be broken down to a few blocks and/or other blocks may be added. Furthermore, in some cases, the blocks can be performed in a different order than described herein. It is to be further noted that some of the blocks are optional. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A system, the system comprising: a controller; and a memory configured to store data used by the controller, the controller being configured to:
(a) provide at least one model indicative of a relationship between first values of one or more physiological parameters of a group of one or more persons and a first probability of the persons within the group to get motion sickness within one or more given time periods;
(b) obtain, from one or more sensors, second values of the physiological parameters, the second values being readings obtained by the sensors from one or more given persons;
(c) estimate, based on the second values and on the model, a second probability of each of the given persons to get motion sickness; and
(d) provide one or more instructions to perform one or more motion sickness prevention measures for at least one of the given persons upon the second probability of the at least one of the given persons exceeding a threshold, to at least reduce the second probability.

2. The system of claim 1, wherein the controller is further configured to repeat steps (b) to (d) a plurality of times over a time period.

3. The system of claim 1, wherein the controller is further configured to perform the motion sickness prevention measures, thereby at least reducing the second probability.

4. The system of claim 1, wherein the motion sickness prevention measures are selected based on results of motion sickness prevention measures previously performed for the at least one of the given persons associated with the second probability exceeding the threshold.

5. (canceled)

6. (canceled)

7. The system of claim 1, wherein the given persons are vehicle passengers in a vehicle driving along a driving route.

8. The system of claim 7, wherein the estimate is also based on at least one of: the driving route of the vehicle, driving conditions along the driving route of the vehicle, a current speed of the vehicle, a predicted speed of the vehicle, a temperature within the vehicle, a type of vehicle, or information of accelerations of the vehicle in at least one axis.

9. (canceled)

10. (canceled)

11. The system of claim 1, wherein the given persons are consumers of Virtual Reality (VR) content wearing a VR headset.

12. (canceled)

13. The system of claim 1, wherein the at least one model is person-specific or one or more of: gender specific, race-specific, or age-range specific.

14. The system of claim 1, wherein the physiological parameters include at least one, or a combination of two or more of: heart rate, heart rate variability, sweat level, respiration rate, skin color, pupil dilation, blood pressure, muscle tension, body temperature, eye gaze movement, microexpression, body movement, electroencephalography (EEG) readings, voice phonation, voice pitch, voice loudness, or voice rate.

15. The system of claim 1, wherein the sensors include at least one of: one or more contact sensors, or one or more proximity or remote sensors such as, but not limited to: lasers, radars, cameras, infrared (IR) sensors, Terahertz radiation sensors, ultrasound sensors, or acoustic sensors.

16-22. (canceled)

23. A method, comprising:
(a) providing at least one model indicative of a relationship between first values of one or more physiological parameters of a group of one or more persons and a first probability of the persons within the group to get motion sickness within one or more given time periods;
(b) obtaining, from one or more sensors, second values of the physiological parameters, the second values being readings obtained by the sensors from one or more given persons;
(c) estimating, based on the second values and on the model, a second probability of each of the given persons to get motion sickness; and
(d) providing one or more instructions to perform one or more motion sickness prevention measures for at least one of the given persons upon the second probability of the at least one of the given persons exceeding a threshold, to at least reduce the second probability.

24. The method of claim 23, further comprising:
repeating steps (b) to (d) a plurality of times over a time period.

25. The method of claim 23, further comprising:
performing the motion sickness prevention measures, thereby at least reducing the second probability.

26. The method of claim 23, wherein the motion sickness prevention measures are selected based on results of motion sickness prevention measures previously performed for the at least one of the given persons associated with the second probability exceeding the threshold.

27. (canceled)

28. (canceled)

29. The method of claim 23, wherein the given persons are vehicle passengers in a vehicle driving along a driving route.

30. The method of claim 29, wherein the estimating is also based on at least one of: the driving route of the vehicle, driving conditions along the driving route of the vehicle, a current speed of the vehicle, a predicted speed of the vehicle, a temperature within the vehicle, a type of vehicle, or information of accelerations of the vehicle in at least one axis.

31. (canceled)

32. (canceled)

33. The method of claim 23, wherein the given persons are consumers of Virtual Reality (VR) content wearing a VR headset.

34. (canceled)

35. (canceled)

36. The method of claim 23, wherein the physiological parameters include at least one, or a combination of two or more of: heart rate, heart rate variability, sweat level, respiration rate, skin color, pupil dilation, blood pressure, muscle tension, body temperature, eye gaze movement, microexpression, body movement, electroencephalography (EEG) readings, voice phonation, voice pitch, voice loudness, or voice rate.

37. The method of claim 23, wherein the sensors include at least one of: one or more contact sensors, or one or more proximity or remote sensors such as, but not limited to: lasers, radars, cameras, infrared (IR) sensors, Terahertz radiation sensors, ultrasound sensors, or acoustic sensors.

38-44. (canceled)

45. A non-transitory computer readable storage medium having computer readable program code embodied therewith, the computer readable program code, executable by a controller of a computer to perform a method of:
(a) providing at least one model indicative of a relationship between first values of one or more physiological parameters of a group of one or more persons and a first probability of the persons within the group to get motion sickness within one or more given time periods;
(b) obtaining, from one or more sensors, second values of the physiological parameters, the second values being readings obtained by the sensors from one or more given persons;
(c) estimating, based on the second values and on the model, a second probability of each of the given persons to get motion sickness; and
(d) providing one or more instructions to perform one or more motion sickness prevention measures for at least one of the given persons upon the second probability of the at least one of the given persons exceeding a threshold, to at least reduce the second probability.

46. (canceled)

47. (canceled)
